(19) United States
(12) Patent Application Publication
(10) Pub. No.: US 2012/0328717 A1
(43) Pub. Date: Dec. 27, 2012

(54) PHARMACEUTICAL KIT FOR TREATING NEURONAL DAMAGES

(76) Inventors: Kuen Jer TSAI, Tainan City (TW); Liang Chao WANG, Tainan City (TW)

(21) Appl. No.: 13/530,259

(22) Filed: Jun. 22, 2012

Related U.S. Application Data
(60) Provisional application No. 61/500,695, filed on Jun. 24, 2011.

(51) Int. Cl. A61K 33/06; A61P 25/00

(52) U.S. Cl. 424/682

(57) ABSTRACT

A pharmaceutical kit for treating neuronal damages is disclosed. The pharmaceutical composition of the present invention comprises: a first pharmaceutical composition comprising a first effective amount of a Mg²⁺-containing compound; and a second pharmaceutical composition comprising a second effective amount of a COX-2 inhibitor.

### CROSS REFERENCE TO RELATED APPLICATION

This application claims the benefit of filing date of U.S. Provisional Application Ser. No. 61/500,695, entitled “Magnesium Sulfate and Nimesulide Have Synergistic Effects on Rescuing Brain Damage after Temporary Focal Ischemia” filed Jun. 24, 2011 under 35 USC §119(e)(1).

### BACKGROUND OF THE INVENTION

1. Field of the Invention

The present invention relates to a pharmaceutical kit for treating neuronal damages and, especially to a pharmaceutical kit for treating ischemic strokes.

2. Description of Related Art

Ischemic stroke is a common disease that causes both death and physical disability, which influence daily lives of patients and result in inconveniences. Although a lot of clinical efforts and resources have been put in to the development of neuronal protective agents to treat brain damages, the effects have been limited. Hence, there are still no ideal agents for treating neuronal damages, not to mention for ischemic strokes.

Generally, one of the reasons that causes neuronal damages is ischemia. After brain ischemia, a series of irreversible reactions take place on the nerve tissue, and thereby magnify neuronal damages. These irreversible reactions include disorders of ion-concentration regulations, activated toxicity, oxidative stress and apoptosis. In addition, these irreversible reactions may lead to the death of brain cells or nerve cells during brain ischemia.

The drugs that are currently used for treating brain ischemia include: metabolism reactivators, anticoagulants, antiplatelet agents, vasodilators, intracranial pressure reducing agents, and hypotensive agents. However, these drugs still have their limitation. For example, metabolism reactivators can increase oxygen usage under brain ischemia, however the effect is not significant enough that it is gradually eliminated from clinical practices. Although anticoagulants can prevent coagulation, a side effect of bleeding may occur. Hence, dangers thereof are higher than other drugs. In addition, although vasodilators can facilitate blood circulations, they cannot be used to expand brain vessels. Furthermore, hypotensive agents can be used only for stroke prevention, but not for stroke treatment.

Since strokes greatly influence human health, it is desirable to develop agents with low side effects but high efficacy that can be applied to treat neuronal damages from strokes.

### SUMMARY OF THE INVENTION

An object of the present invention is to provide a pharmaceutical kit for treating neuronal damages and myocardial infarctions.

Another object of the present invention is to provide a method for treating neuronal damages and myocardial infarctions.

The pharmaceutical kit of the present invention comprises: a first pharmaceutical composition comprising a first effective amount of a Mg²⁺-containing compound; and a second pharmaceutical composition comprising a second effective amount of a cyclooxygenase-2 (COX-2) inhibitor.

According to the pharmaceutical kit of the present invention, the Mg²⁺-containing compound and the COX-2 are prepared as two different formulations for separate administrations. Hence, according to the pharmaceutical kit of the present invention, treatment for neuronal damages is performed by administering a combination of Mg²⁺-containing compound and the COX-2 inhibitor. The Mg²⁺-containing compound has the effect of neural protection. In addition, the COX-2 inhibitor can inhibit inflammatory response from occurring when blood flows back to brain, and ultimately treat neuronal damages. Hence, the combination treatment of the present invention is effective in treating neuronal damages, and especially ischemic strokes.

Preferably, the Mg²⁺-containing compound used in the pharmaceutical kit of the present invention is MgSO₄. In addition, according to the pharmaceutical kit of the present invention, the COX-2 inhibitor can be nimesulide or meloxicam. The chemical names of nimesulide and meloxicam are N-(4-Nitro-2-phenoxyphenyl)methane-sulfonamide and 4-hydroxy-2-methyl-N-(5-methyl-2-thiazolyl)-2H-1,2-benzothiazine-3-carboxamide-1,1-dioxide, respectively. Preferably, the COX-2 inhibitor is nimesulide. Clinically, MgSO4 is used for pre-eclampsia in pregnancy, and the COX-2 is widely used as an anti-inflammatory drug.

According to the pharmaceutical kit of the present invention, neuronal damages may include brain ischemia, stroke, spinal cord injury, traumatic brain injury, peripheral nerve ischemia, or myocardial infarction. Preferably, the neuronal damage is ischemia stroke.

In addition, according to the pharmaceutical composition of the present invention, the first effective amount of the Mg²⁺-containing compound cannot be more than 90 mg/Kg. Preferably, the first effective amount thereof is 20-60 mg/Kg. More preferably, the first effective amount thereof is 40-50 mg/Kg. When the dosage of the Mg²⁺-containing compounds such as MgSO₄ is more than 90 mg/Kg, there exists a risk of cardiovascular depression. In addition, the second effective amount of the COX-2 inhibitor cannot be more than 12 mg/Kg, which is the daily dosage in clinical practice. Preferably, the second effective amount thereof is 2-12 mg/Kg. More preferably, the second effective amount thereof is 3-7 mg/Kg. When the dosage of the COX-2 inhibitor is more than 12 mg/Kg, risks of adverse drug reactions such as digestive system damages, hepatotoxicity/renal toxicity and cardiovascular diseases may be increased. In the present invention, the term “mg/Kg” means a dosage (mg) administered to a subject per kilogram (Kg).

The pharmaceutical kit for treating neuronal damages of the present invention may further comprise a pharmaceutically acceptable carrier. Herein, the pharmaceutically acceptable carrier can be respectively formulated with the Mg²⁺-containing compound and/or the COX-2 inhibitor to form a formulation. More specifically, the first pharmaceutical composition of the present invention comprises: the Mg²⁺-containing compound, and selectively a pharmaceutically acceptable carrier formulated with the Mg²⁺-containing compound to form a single formulation. In addition, the second pharmaceutical composition comprises: the COX-2 inhibitor, and selectively a pharmaceutically acceptable carrier formulated with the COX-2 inhibitor to form another single formulation. According to the pharmaceutical kit of the present invention, the term “pharmaceutically acceptable carrier” means that the carrier must be compatible with the active ingredients (and preferably, capable of stabilizing the active ingredients) and not be deleterious to the subject to be treated. The carrier may be at least one selected from the group consisting of active agents, adjuvants, dispersants, wetting agents and suspending agents. The example of the carrier may be microcrystalline cellulose, mannitol, glucose, non-Tat milk powder, polyethylene, polyvinylprrolidone, starch or a combination thereof.

In addition, according to the pharmaceutical kit of the present invention, the term “acceptable” means that that the carrier must be compatible with active ingredients (and preferably, capable of stabilizing the active ingredients) and not be deleterious to the subject to be treated. The term “treating” used in the present invention refers to the application or administration of the pharmaceutical composition or the pharmaceutical kit to a subject with symptoms or tendencies of neuronal damages in order to cure, heal, alleviate, relieve, alter, remedy, ameliorate, improve, or affect the symptoms or tendencies of neuronal damages. Furthermore, “an effective amount” used herein refers to the amount of each active ingredients required to confer therapeutic effect on the subject. The effective amount may vary according to the route of administration, excipient usage, and co-usage with other active ingredients.

The present invention further provides a method for treating neuronal damages, which comprises the following steps: administering a first pharmaceutical composition comprising a first effective amount of a Mg²⁺-containing compound in the aforementioned pharmaceutical kit to a subject in need thereof; and administering a second pharmaceutical composition comprising a second effective amount of a COX-2 inhibitor in the aforementioned pharmaceutical kit to the subject. Preferably, the Mg²⁺-containing compound is administered followed by the occurrence of ischemic stroke and before thrombolysis. More preferably, the Mg²⁺-containing compound is administered within 30 min of ischemic stroke. Most preferably, the Mg²⁺-containing compound is administered immediately after ischemic stroke. In addition, the COX-2 inhibitor is preferably administered before thrombolysis. More preferably, the COX-2 inhibitor is administered within 3 hr of ischemic stroke. According to the statistical data, thrombolysis generally occurs after 3 hr of the stroke. More preferably, the COX-2 inhibitor is administered within 3 hr of ischemia stroke, and the second dose of the COX-2 inhibitor is administered within 24 hr of the first administration.

The pharmaceutical kit and the method for treating neuronal damages of the present invention can be administered via parenteral, inhalation, local, retal, nasal, sublingual, or vaginal delivery, or implanted reservoir. Herein, the term “parenteral delivery” includes subcutaneous, intradermic, intravenous, intraarticular, intra-arterial, synovial, intrapleural, intrathecal, local, and intracranial injections. Preferably, the Mg²⁺-containing compound is administered via parenteral delivery, and the COX-2 inhibitor is administered via oral or parenteral delivery.

Other objects, advantages, and novel features of the invention will become more apparent from the following detailed description when taken in conjunction with the accompanying drawings.

### BRIEF DESCRIPTION OF THE DRAWINGS

FIG. 1 is a result of infarct volume evaluation according to Experiment 1 of the present invention;

FIG. 2A is a scattergram of neurological severity score after 24 hr of MCAO according to Experiment 1 of the present invention;

FIG. 2B is a scattergram of neurological severity score after 72 hr of MCAO according to Experiment 1 of the present invention;

FIG. 3 is a result of infarct volume evaluation according to Experiment 2 of the present invention;

FIG. 4 is a result of infarct volume evaluation according to Experiment 3 of the present invention; and

FIG. 5 is a result of infarct volume evaluation according to Experiment 4 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Middle Cerebral Artery Occlusion

150 Sprague-Dawley rats (SD rats) weighing 220-250 gm were purchased from the Laboratory Animal Center of National Chung Kung University.

In the present experiments, middle cerebral artery occlusion (MCAO) known in the art was performed on SD rats to establish animal models with ischemic strokes by using occluding intra-luminal suture. The process for performing MCAO is briefly described as follows.

A segment of 4-O nylon with blunted tip was inserted through the right internal carotid artery to occlude blood flow of middle cerebral artery (MCA). Laser Doppler flowmeter was used to monitor changes of cerebral blood flows in the MCA territory. When the laser Doppler signal was reduced more than 70% of baseline, it indicated that MCAO models were successfully established. Anesthetic time was limited to 20 minutes to minimize the influence of isoflurane on the outcome of MCAO. After recovery from anesthesia, rats without body asymmetry were regarded as failed MCAO models and excluded from further experiments.

After MCAO was established for 90 min, intra-luminal occluder was removed to re-perfuse the brain. When the laser Doppler signal was recovered to over 60% of the baseline, it indicated that the reperfusion was successful.

### Drugs

The standard powder of nimesulide used in the present experiment was provided by Lotus Pharmaceutical CO., LTD. Taiwan, R.O.C, and the dosage thereof was 15 mg/1.5 ml in an injection formulation.

2% of polyvinylpyrrolidone (PVP) (Sigma Chemical Co., St Louis, Mo., USA) was used as a vehicle, and the standard powder of nimesulide was dissolved therein for further intra-peritoneal injection.

10 wt % of MgSO₄ (Taiwan Biotech CO., LTD. Taiwan, R.O.C.) was diluted to 5 wt % with normal saline.

Meloxicam was diluted 10 times with distilled water for further intra-peritoneal injection.

In the following experiments, normal saline and 2 wt % of PVP were used as placebos.

### Experiment 1

SD rats were randomly assigned to one of the following groups, each group consisting of 30 SD rats (n=30): SVe group treated with placebo (i.e. normal saline and 2 wt % of PVP), MgVe group treated with 45 mg/kg of MgSO₄ and 2 wt % of PVP, SNi group treated with normal saline and 6 mg/kg of nimesulide, and MgNi group treated with 45 mg/kg of MgSO₄ and 6 mg/kg of nimesulide. In each group, MgSO₄ or normal saline were injected intravenously immediately after MCAO, and nimesulide or PVP were injected intra-peritoneally just before reperfusion.

Five rats in each group were used for infarct volume evaluation, brain edema evaluation and other analyses.

### Evaluation of Infarct Volume

Quantification of infarct volume using 2,3,5-triphenyltetrazolium chloride (TTC) stain was performed with conventional methods provided by Lin et al. (Lin T N, He Y Y, Wu G, Khan M, Hsu C Y, Effect of brain edema on infarct volume in a focal cerebral ischemia model in rats. Stroke 1993; 24: 117-121.) and Swanson et al. (Swanson R A, Morton M T, Tsao-Wu G, et al. A semi-automated method for measuring brain infarct volume. J Cereb Blood Flow Metab 1990; 10: 290-293.).

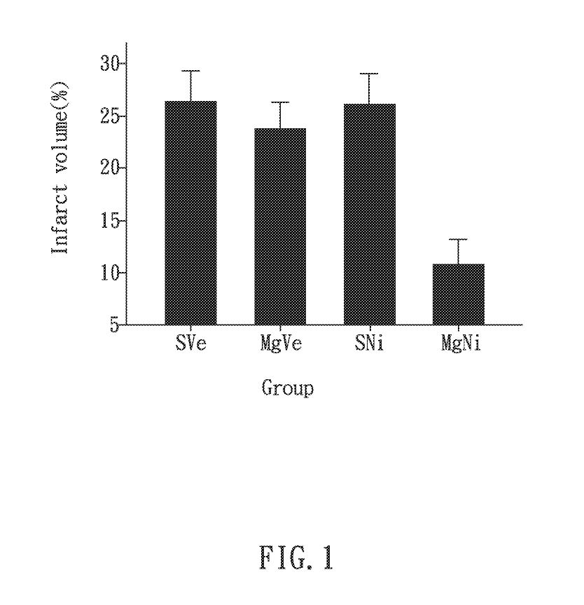

When SD rats in each group were treated with MCAO for 90 min and reperfusion for 72 hr, infarct volumes were evaluated by TTC staining. The results are shown in FIG. 1. Compared to SVe group, a decrease of infarct volume was not observed in MgVe group treated with a single dose of MgSO₄. A single dose of nimesulide also had no neuro-protective effect in SNi group. In addition, the infarction volume was 10.93±6.54% in MgNi group (p=0.007, Dunnett t test), compared to that of 26.43±17.80% in SVe group. Furthermore, as shown in FIG. 1, there was a significant decrease of infarction volume in MgNi group, compared to MgVe group and SNi group.

These results indicated that a significant suppression of infarction volume can be accomplished by the combination treatment of MgSO₄ and nimesulide. Hence, the pharmaceutical kit of the present invention can inhibit ischemia damages after occlusion.

### Evaluation of Brain Edema

In general, blood brain barrier achieves maximum disruption 48 hr after stroke, then brain edema is observed. Herein, cerebral edema was determined by measuring the brain water content with the wet-dry method after MCAO was established for 48 hr. The wet-dry method was provided by Hatashita et al. (Hatashita S, Hoff J T, Salamat S M. Ischemic brain edema and the osmotic gradient between blood and brain, J Cereb Blood Flow Metab 1998; 8, 552-559.).

The difference of water content in ipsilateral and contralateral hemisphere (Δ% of water content) 48 hr after ischemia-reperfusion injury was evaluated with the wet-dry method. After MCAO was established and a reperfusion was performed for 48 hr, combined treatment with MgSO₄ and nimesulide (MgNi group) can significantly inhibit post-ischemia brain edema (p=0.023, Dunnett t test), compared to SVe group. However, the inhibited post-ischemia brain edema cannot be Observed in the group treated with MgSO₄, or nimesulide alone.

Hence, the occurrence of post-ischemia brain edema can be decreased and the disruption of blood brain barrier can further be inhibited by combination treatment of MgSO₄ and nimesulide.

### Neuro-Behavioral Test

Neurological outcome were evaluated using a neurological severity scoring (NSS) system, which is provided by Clark et al (Clark W M, Rinker L G, Lessov N S, et al. Lack of interleukin-6 expression is not protective against focal central nervous system ischemia. Stroke 2000; 31: 1715-1720.).

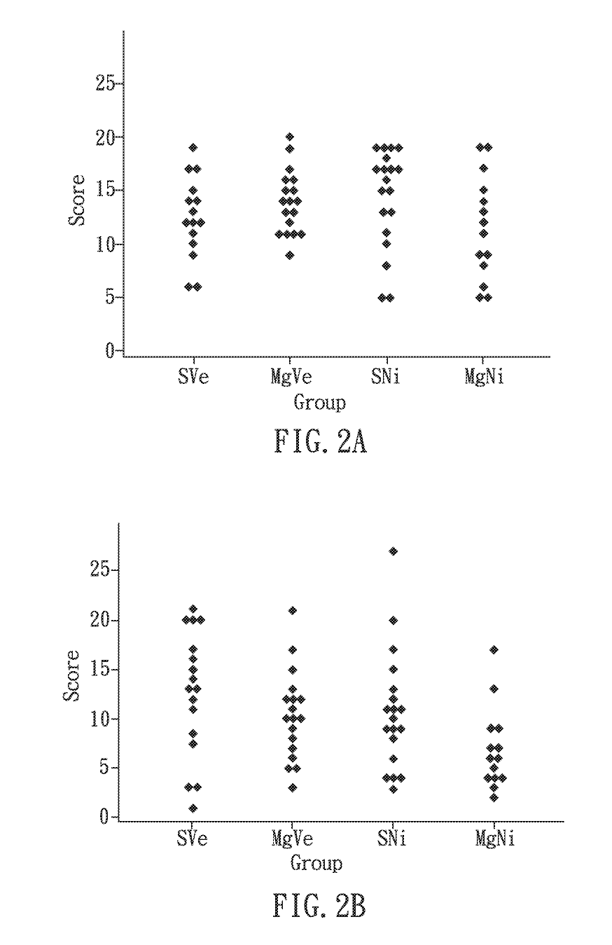

Scattergrams of neurological scores of each group are shown in FIG. 2A and FIG. 2B, wherein the longitudinal axis thereof represents the scores evaluated by the neurological severity scoring (NSS) system.

The perfect score in the present test was 28 scores, and the scores were evaluated according to the performance of SD rats including steps, behavior symmetry, walking direction, behavior on body rotation, front leg extension, rotation tendency after stimulation, and perception reaction. Observers evaluated each item from 0 score to 4 score, and the sum of the scores of each item was the total score. High total score indicated poor performance. As shown in FIG. 2A, the neurological severity scores were not significantly different in each group after MCAO and reperfusion for 24 hr. However, after MCAO and reperfusion for 72 hr, the MgNi group treated with MgSO₄ and nimesulide showed significantly reduced neurological deficit, compared to SVe group, MgVe group and SNi group.

These results indicate that the brain damages caused by ischemia can be alleviated and the neurological deficits can be inhibited by combination treatment of MgSO₄ and nimesulide.

### Experiment 2

SD rats were randomly assigned to one of the following groups: SVe group treated with placebo (i.e. normal saline and 2 wt % of PVP); SNi group treated with normal saline after MCAO and 6 mg/kg of nimesulide before perfusion (90 min after MCAO); 90 min group, 60 min group and 30 min group treated with 45 mg/kg of MgSO₄ after 90 min, 60 min and 30 min of MCAO respectively, and then treated with 6 mg/kg of nimesulide before perfusion (90 min after MCAO); and MgNi group treated with 45 mg/kg of MgSO₄ immediately after MCAO and then 6 mg/kg of nimesulide before perfusion (90 min after MCAO).

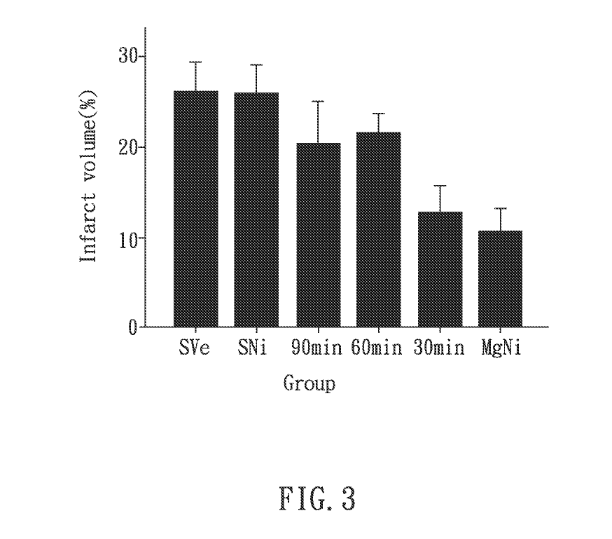

The same method as described in Experiment 1 was performed in the present experiment to evaluate the infarct volume of rats in each group. The result is shown in FIG. 3.

As shown in FIG. 3, a synergistic effect of MgSO₄ and nimesulide can be observed in the 30 min group, wherein SD rats were treated with MgSO₄ within 30 mm of stroke (MCAO treatment). In addition, the neuro-protective effect of the 30 min group was almost the same as that of the MgNi group that MgSO₄ was immediately administered after stroke.

### Experiment 3

SD rats were randomly assigned to one of the following groups: SNi6X2 group treated with normal saline after MCAO and 6 mg/kg of nimesulide before perfusion (90 min after MCAO), and 6 mg/kg of nimesulide after 24 hr of perfusion; and Mg45Ni6X2 group treated with 45 mg/kg of MgSO₄ and 6 mg/kg of nimesulide before perfusion (90 min after MCAO), and 6 mg/kg of nimesulide after 24 hr of perfusion.

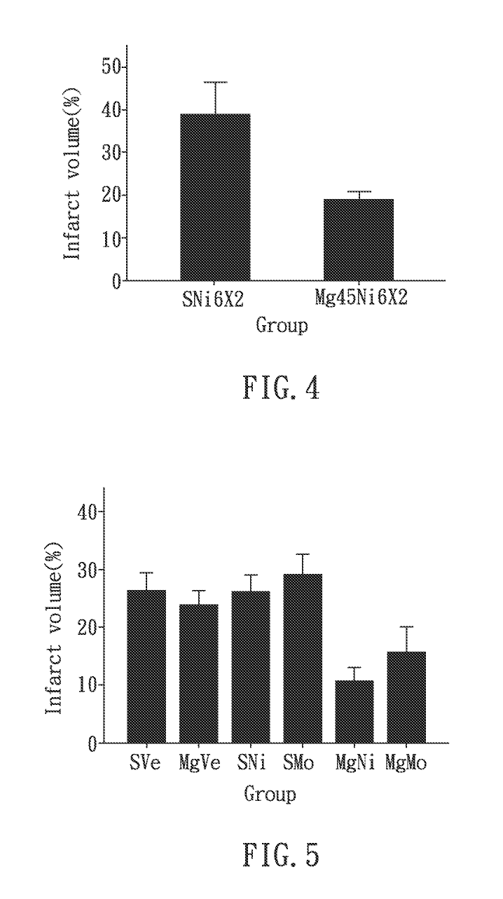

The same method as described in Experiment 1 was performed in the present experiment to evaluate the infarct volume of rats in each group. The result is shown in FIG. 4.

As shown in FIG. 4, even when MgSO₄ cannot be provided immediately after stroke, the neuro-protective effect still can be obtained when MgSO₄ and nimesulide were provided together 90 min after stroke (MCAO treatment) with an additional dose of nimesulide provided after 24 hr.

### Experiment 4

SD rats were randomly assigned to one of the following groups: SW group treated with placebo (i.e. normal saline and 2 wt % of PVP), MgVe group treated with 45 mg/kg of MgSO₄ and 2 wt % of PVP, SNi group treated with normal saline and 6 mg/kg of nimesulide, MgNi group treated with 45 mg/kg of MgSO₄ and 6 mg/kg of nimesulide, MgMo group treated with 45 mg/kg of MgSO₄ and 1.25 mg/kg of meloxicam, and SMo group treated with normal saline and 1.25 mg/kg of meloxicam. In each group, MgSO₄ or normal saline were injected intravenously immediately after MCAO, and nimesulide, meloxicam or PVP were injected intra-peritoneally just before reperfusion.

The same method as described in Experiment 1 was performed in the present experiment to evaluate the infarct volume of rats in each group. The result is shown in FIG. 5.

These results indicated that a significant suppression of infarction volume was accomplished by combination treatment of MgSO₄ and meloxicam (MgMo group), compared to treatment of meloxicam alone (SMo group). In addition, the combination treatment effect of MgSO₄ and meloxicam (MgMo group) on suppression of infarction volume is similar to that of MgSO₄ and nimesulide (MgNi group).

According to the results in Experiments 1 and 2, the infarction and neuronal damages caused by temporary focal ischemia can be reduced and neuro-protective effects can be accomplished by the combination treatment of MgSO₄ and a COX-2 inhibitor such as nimesulide and meloxicam, even if the doses of MgSO₄ and the COX-2 inhibitor are low.

Although the present invention has been explained in relation to its preferred embodiment, it is to be understood that many other possible modifications and variations can be made without departing from the spirit and scope of the invention as hereinafter claimed.

What is claimed is:

1. A pharmaceutical kit for treating neuronal damages, comprising:
a first pharmaceutical composition comprising a first effective amount of a Mg²⁺-containing compound; and
a second pharmaceutical composition comprising a second effective amount of a COX-2 inhibitor.

2. The pharmaceutical kit as claimed in claim 1, wherein the Mg²⁺-containing compound is MgSO₄.

3. The pharmaceutical kit as claimed in claim 1, wherein the COX-2 inhibitor is nimesulide or meloxicam.

4. The pharmaceutical kit as claimed in claim 1, wherein the COX-2 inhibitor is N-(4-Nitro-2-phenoxyphenyl)methane-sulfonamide, or 4-hydroxy-2-methyl-N-(5-methyl-2-thiazolyl)-2H-1,2-benzothiazine-3-carboxamide-1,1-dioxide.

5. The pharmaceutical kit as claimed in claim 1, wherein the neuronal damages include brain ischemia, stroke, spinal cord injury, traumatic brain injury, peripheral nerve ischemia, or myocardial infarction.

6. The pharmaceutical kit as claimed in claim 2, wherein the first effective amount is 20-60 mg/Kg.

7. The pharmaceutical kit as claimed in claim 6, wherein the first effective amount is 40-50 mg/Kg.

8. The pharmaceutical kit as claimed in claim 3, wherein the second effective amount is 2-12 mg/Kg.

9. The pharmaceutical kit as claimed in claim 8, wherein the second effective amount is 3-7 mg/Kg.
